(19) United States
(12) Patent Application Publication
(10) Pub. No.: US 2022/0250822 A1
(43) Pub. Date: Aug. 11, 2022

(54) DISPENSING RECEPTACLE AND BAG APPARATUS

(71) Applicant: RONEN RIMON, JERUSALEM (IL)

(72) Inventor: RONEN RIMON, JERUSALEM (IL)

(21) Appl. No.: 17/623,671

(22) PCT Filed: Aug. 17, 2020
(86) PCT No.: PCT/IL2020/050903

§ 371 Date: Dec. 29, 2021

(30) Foreign Application Priority Data
Sep. 1, 2019 (IL) 269047

(51) Int. Cl. B65D 77/06; B65D 47/06; B65D 83/00; B65D 85/72

(52) U.S. Cl. CPC B65D 77/06; B65D 85/72; B65D 83/00; B65D 47/06

(57) ABSTRACT

A dispensing receptacle (10), including: a frame member (12A); a bag and lid member (12D), for being connected to the frame member (12A), the bag and lid member (12D) including a bag (18) for containing content (24), and an orifice (16) for dispensing the content (24) therethrough, thereby the content (24) does not dirty the frame member (12A).

### TECHNICAL FIELD

The invention relates to the field of containers, for dispensing therefrom.

### BACKGROUND

Non-disposable pressable bottles are used, for dispensing sauces. At the end of the use it must be cleaned for refilling, being disadvantaged.

One known solution is a disposable bag. However, yet, the outer screw-on area of the bottle is exposed and may become dirty when refilled.

There is a long felt need to provide a solution to the above-mentioned and other problems of the prior art.

### SUMMARY

A dispensing receptacle, including:
a frame member; and
a bag and lid member, for being connected to the frame member, the bag and lid member including a bag.

### BRIEF DESCRIPTION OF THE DRAWINGS

Embodiments, features, and aspects of the invention are described herein in conjunction with the following drawings:

FIG. 1 depicts a dispensing receptacle according to one embodiment of the invention, at a non-assembled state.

FIG. 2 depicts the first and second steps of using the dispensing receptacle of FIG. 1.

FIG. 3 depicts the third and fourth steps of using the dispensing receptacle of FIG. 1.

FIG. 4 magnifies a portion of FIG. 3.

FIG. 5 depicts the fifth step of using the dispensing receptacle of FIG. 1.

FIG. 6 depicts the bag apparatus of FIG. 1 alone.

FIG. 7 depicts the bag apparatus of FIG. 1 including the lid, for being supplied including the sauce.

FIG. 8 depicts the dispensing receptacle at the non-assembled state of FIG. 1 according to other embodiments.

### DETAILED DESCRIPTION

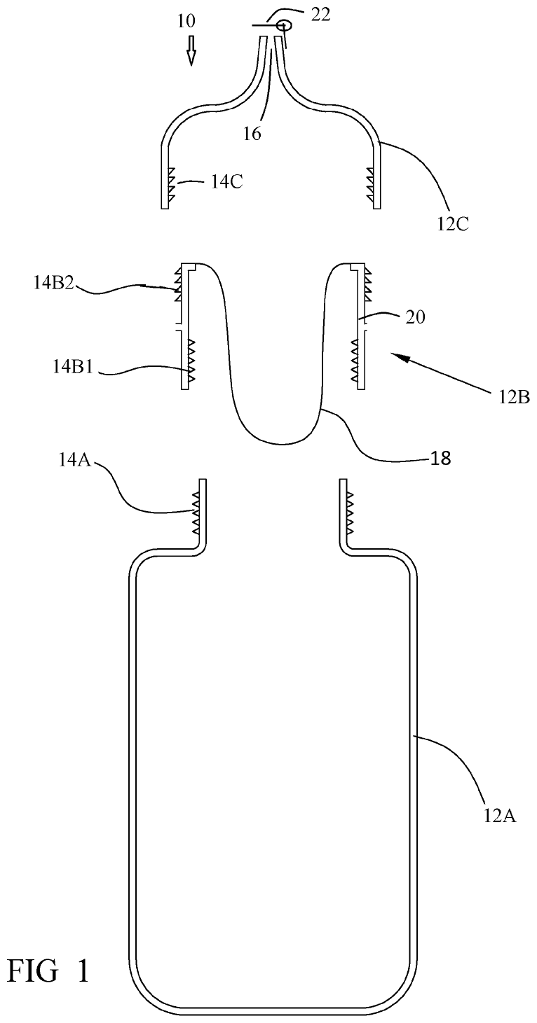

FIG. 1 depicts a dispensing receptacle according to one embodiment of the invention, at a non-assembled state.

A dispensing receptacle 10 according to one embodiment of the invention, includes a frame member 12A; a lid member 12C, for dispensing from an orifice 16 thereof and a bag apparatus 12B including a rack 20 for a bag 18, the rack for being disposed between frame member 12A and lid member 12C.

Frame member 12A is intended to be non-disposable, whereas bag apparatus 12B is intended to be disposable, for separating the content, such as a sauce, from non-disposable frame member 12A, for maintaining frame member 12A clean.

Frame member 12A includes a thread 14A, for interlacing a thread 14B1 of rack 20 of bag apparatus 12B; and lid member 12C includes a connecting member 14C, such as a thread, for connecting to connection member 14B2, such as a thread, of rack 20 of bag apparatus 12B.

Bag apparatus 12B includes a bag 18, being irremovable from rack 20 including thread 14B1 and connection member 14B2, for instance being soldered, stuck or cast thereto.

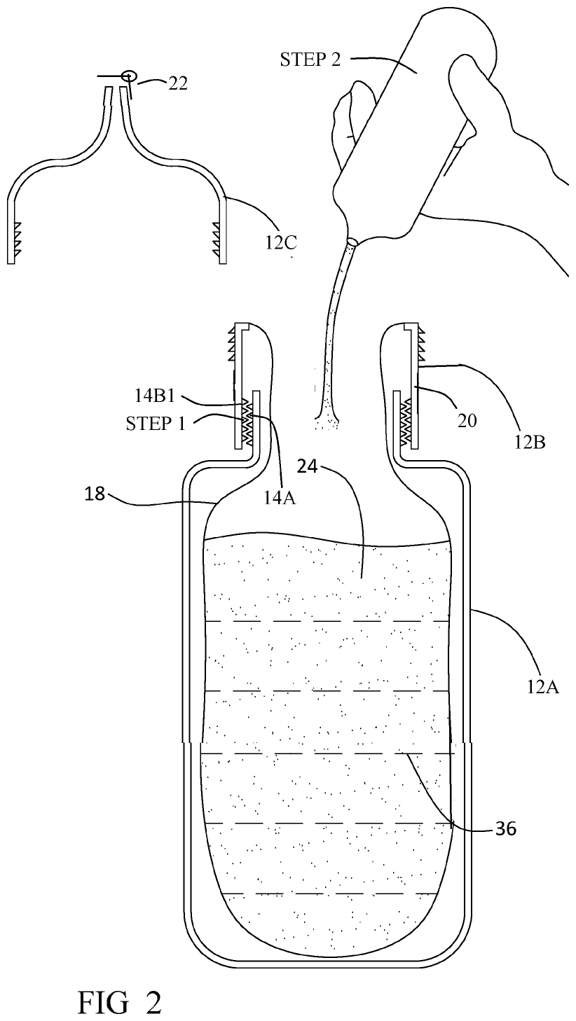

FIG. 2 depicts the first and second steps of using the dispensing receptacle of FIG. 1.

At the first step, the user connects rack 20 of bag apparatus 12B to frame member 12A via thread 14A of frame member 12A being threaded to thread 14B1 of rack 20 of bag apparatus 12B.

At the second step, sauce 24 is poured into bag 18 of bag apparatus 12B.

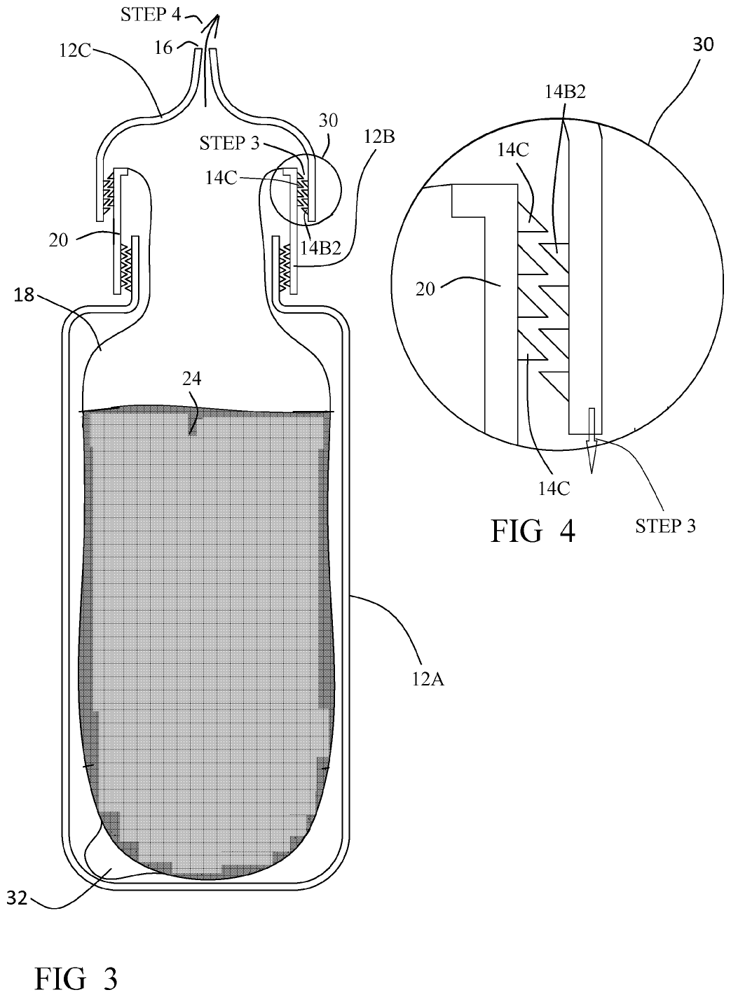

FIG. 3 depicts the third and fourth steps of using the dispensing receptacle of FIG. 1.

At the third step, lid member 12C is connected to rack 20 of bag apparatus 12B via connecting members 14C and 14B2.

At the fourth step, sauce 24 may be dispensed through an orifice 16 of lid member 12C.

FIG. 4 magnifies a portion of FIG. 3.

Connecting members 14C and 14B2 may be configured to allow connecting only, while not allowing separation.

This may be applied by connecting members 14C and 14B2 being one-directional elastic cogs, for connecting by pressing one-directional cogs 14C and 14B2 one towards the other, rather than by rotating.

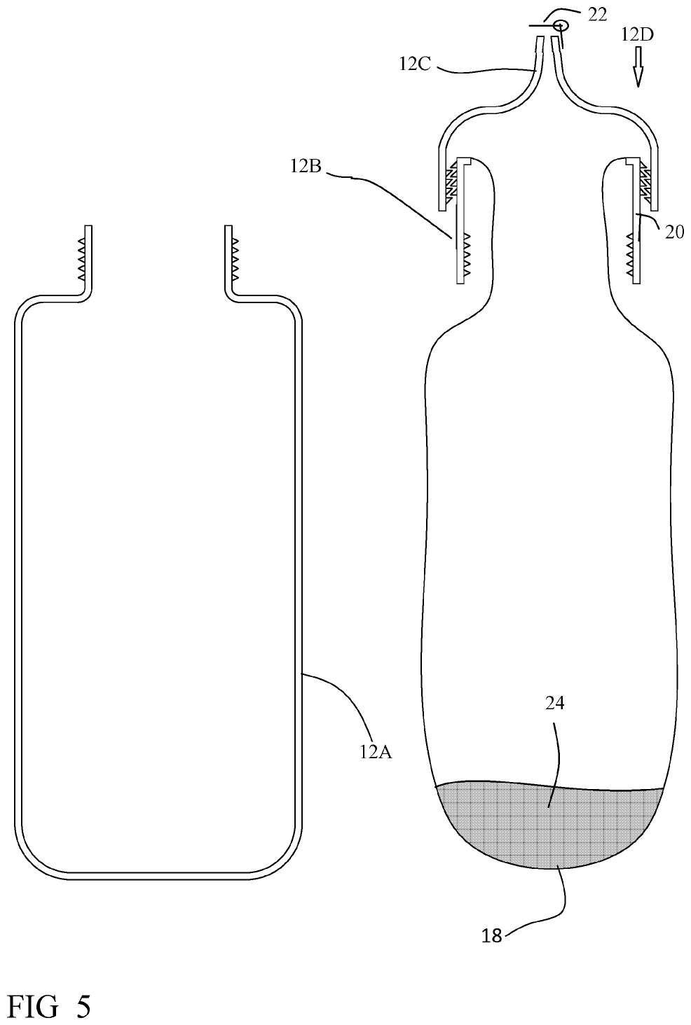

FIG. 5 depicts the fifth step of using the dispensing receptacle of FIG. 1.

At the fifth step, rack 20 of bag apparatus 12B being yet connected to lid member 12C is removed from frame member 12A, leaving it clean, for connecting a new bag apparatus 12B, as described in FIG. 2.

According to another embodiment, bag apparatus 12B and lid member 12C may be marketed as a single member 12D, which may further contain sauce 24.

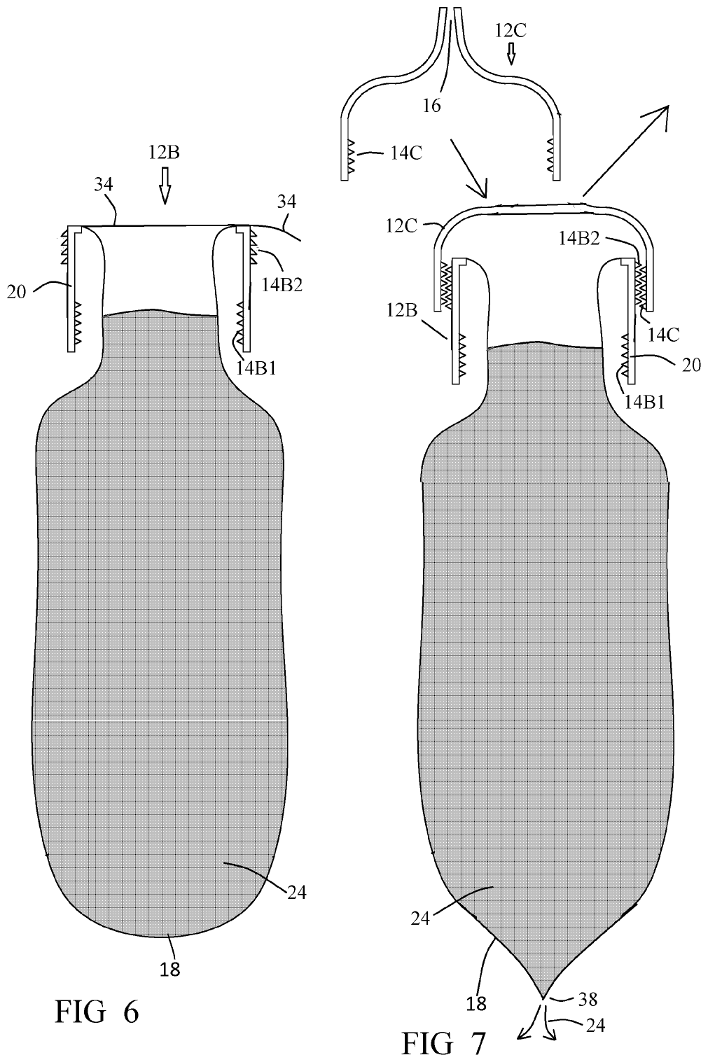

FIG. 6 depicts the bag apparatus of FIG. 1 alone.

Bag apparatus 12B may be supplied including sauce 24, by being closed by a reed 34.

FIG. 7 depicts the bag apparatus of FIG. 1 including the lid, for being supplied including the sauce.

Bag apparatus 12B may be supplied including sauce 24, by being closed by lid member 12C, providing improved closing in relation to reed 34 of FIG. 6, being by dispensing directly from a corner 38 of bag 18 to be punctured.

Connecting members 14B2 and 14C of bag rack 20 may constitute normal threads, for allowing removing lid member 12C lacking an orifice, for replacing it with a lid member 12C including orifice 16, for dispensing therethrough.

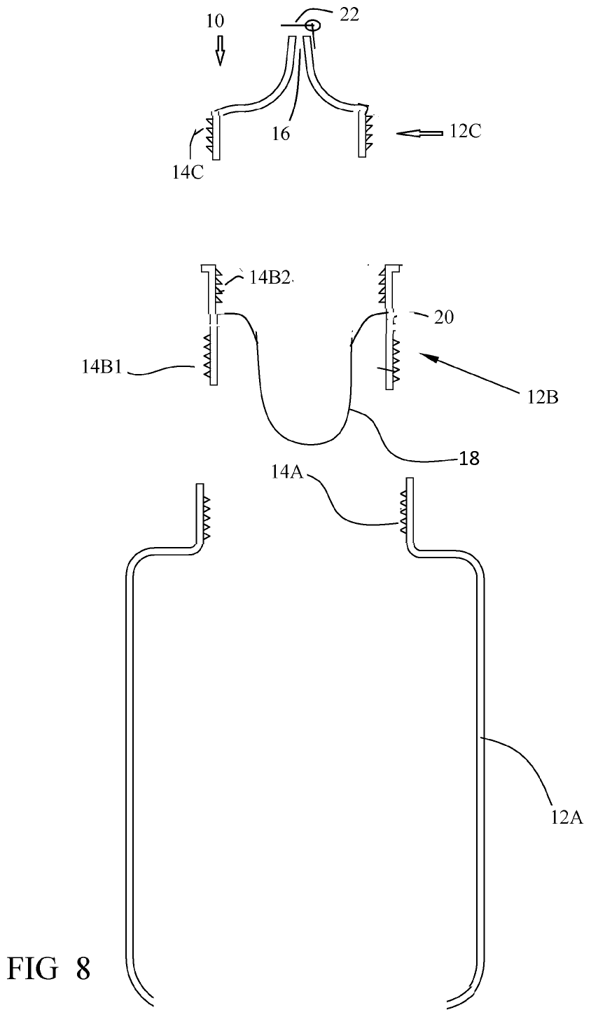

FIG. 8 depicts the dispensing receptacle at the non-assembled state of FIG. 1 according to other embodiments.

Each of threads and connecting members 14A, 14B1,14B2, and 14C may be external or internal, such as of FIG. 8 being different from FIG. 1.

Frame member 12A may be a receptacle as shown in FIG. 1, or a frame only, such as in FIG. 8.
Numeral 10 denotes the dispensing receptacle according to one embodiment of the invention;
12A: frame member;
12B: bag apparatus;
12C: lid member;
12D: bag and lid member;
14A,14B1: threads;
14B2,14C: connection members, such as threads, elastic cogs, etc.;
16: dispensing orifice;
18: bag of bag apparatus 12B;
20: rack for hanging bag 18 of bag apparatus 12B;
22: openable cap of lid member 12C;
24: content, such as a sauce;
30: zone;
32: tearing line in bag 18, for removing remainder of content 24;
38: corner of bag 18 for being pierced;
36: quantity mark.

The reference numbers in the claims should not be interpreted as limiting the claims in any form.

What is claimed is:

1. A dispensing receptacle (10), comprising:
a frame member (12A);
a lid member (12C); and
a bag apparatus (12B), comprising a rack (20) for being connected between said frame member (12A) and said lid member (12C), said bag apparatus (12B) further comprising a bag (18) being irremovable from said rack (20), said bag (18) for containing content (24) to be dispensed through said lid member (12C),
thereby said content (24) does not dirty said frame member (12A).

2. The dispensing receptacle (10) according to claim 1, wherein said frame member (12A) comprises a first thread (14A), and wherein said rack (20) of said bag apparatus (12B) comprises a second thread (14B1), for connecting said first thread.

3. The dispensing receptacle (10) according to claim 1, wherein said lid member (12C) comprises first connecting member (14C), and
wherein said rack (20) of said bag apparatus (12B) comprises a second connecting member (14B2), for connecting to said first connecting member (14C).

4. The dispensing receptacle (10) according to claim 3, wherein said first connecting member (14C) and said second connecting member (14B2) are configured to allow connection therebetween and to not allow separation therebetween.

5. The dispensing receptacle (10) according to claim 3, wherein said first connecting member (14C) and said second connecting member (14B2) comprise one-directional cogs, thereby said connecting is operable by pressing said cogs one towards the other.

6. The dispensing receptacle (10) according to claim 1, wherein said bag (18) is irremobvable from said rack (20) of said bag apparatus (12B), via at least one member selected from a group consisting of sticking, welding, casting.

7. A dispensing receptacle (10), comprising:
a frame member (12A); and
a bag and lid member (12D), for being connected to said frame member (12A), said bag and lid member (12D) comprising a bag (18) for containing content (24), and an orifice (16) for dispensing said content (24) therethrough,
thereby said content (24) does not dirty said frame member (12A).

8. A bag apparatus (12B), comprising:
a rack (20) comprising:
a) thread (14B1), for connecting said bag apparatus (12B) to a frame member (12A);
b) a connection member (14B2), for connecting said bag apparatus (12B) to a lid member (12C); and
a bag (18) being irremovable from said rack (20), said bag (18) for containing content (24) to be dispensed.

9. The bag apparatus (12B) according to claim 8, further comprising:
a reed (34) deployed on said rack (20), for covering said bag (18),
thereby said bag apparatus (12B) alone is configured to contain said content (24).

10. The bag apparatus (12B) according to claim 8, wherein said bag (18) comprises a corner (38),
thereby allowing piercing said corner (38), for dispensing said content (24) therethrough.
